(19) United States
(12) Patent Application Publication
(10) Pub. No.: US 2019/0355477 A1
(43) Pub. Date: Nov. 21, 2019

(54) TEST PANEL ANALYSIS

(71) Applicant: Beckman Coulter, Inc., Brea, CA (US)

(72) Inventors: Wido Menhardt, Los Gatos, CA (US); Martha C. Davis, Brea, CA (US)

(21) Appl. No.: 15/983,235

(22) Filed: May 18, 2018

(51) Int. Cl. G16H 50/70; G06F 7/08; G06F 17/30

(52) U.S. Cl. CPC G16H 50/70; G06F 17/30598; G06F 7/08

(57) ABSTRACT

Aspects of the disclosed technology can be used to implement methods in which a co-occurrence matrix of test types can be transformed through a process which includes sorting based on an eigenvector corresponding to a non-zero eigenvalue, and the transformed matrix can then be used to efficiently identify types of tests with high co-occurrence. Alternative approaches which use modified k-means clusters are also possible and could be applied in similar contexts as approaches using eigenvector sorting.

### FIELD

The disclosed technology pertains to identifying clusters having high co-occurrence, such as groups of diagnostic tests that are performed together at high frequency.

### BACKGROUND

Often, when blood or another body fluid is analyzed, it may typically be subjected to multiple different tests. For some diagnostic tasks or specific diseases there may be recommendations of pre-defined groups of tests (“panels”) that should be run together to ascertain a more complete picture of a patient's condition. However, it is possible that clinicians may have their own preferred panels that could differ from the recommendations, or that they may diverge from recommended panels by ordering tests in an ad-hoc or non-systematic manner. Additionally, there may not always be applicable panel recommendations, and so even a clinician who consistently follows such recommendations when they are available may at times make their own idiosyncratic test orders simply as a result of recommended panels being unavailable. This can cause various problems, such as waste in the event a clinician orders tests that are redundant for each other.

### SUMMARY

There is a need for improved technology for identifying groups of tests that may be run together with a high frequency. It may thus be an object of some embodiments to provide a method that could comprise steps such as obtaining a set of co-occurrence data for each of a plurality of test types, defining a co-occurrence distribution based on co-occurrence data, generating a transformation operator (e.g., a derivative operator such as a Laplacian matrix) based on the co-occurrence distribution, generating a sorting construct based on the transformation operator, generating an evaluation distribution based on sorting the co-occurrence distribution with the sorting construct, and generating a set of co-occurrence clusters for the plurality of types of test based on the evaluation distribution. In some embodiments, this objective may be fulfilled by the subject matter of the independent claims, wherein further embodiments are incorporated in the dependent claims.

### BRIEF DESCRIPTION OF THE DRAWINGS

The drawings and detailed description that follow are intended to be merely illustrative and are not intended to limit the scope of the invention as contemplated by the inventors.

FIG. 1 is an architecture which may be used in some embodiments.

FIG. 2 is an exemplary co-occurrence distribution in the form of a matrix.

FIG. 3 is a flowchart showing a process which may be used in some embodiments to derive an evaluation distribution from a co-occurrence distribution.

FIG. 4 is an exemplary evaluation distribution in the form of a matrix.

FIG. 5 is an exemplary process that may be used to automatically identify co-occurrence clusters in some embodiments.

FIG. 6 is an exemplary process that may be used to automatically identify co-occurrence clusters in some embodiments.

### DETAILED DESCRIPTION

In light of the above, it could be beneficial to be able to identify tests which are often ordered together both to address problems such as waste, as well as for other purposes such as identifying emerging trends in testing. However, with conventional approaches it has not been feasible to identify these types of patterns. According to a first aspect some embodiments may include a method comprising steps such as obtaining a set of co-occurrence data for each of a plurality of test types, defining a co-occurrence distribution based on the co-occurrence data, generating a transformation operator based on the co-occurrence distribution, generating a sorting construct based on the transformation operator, generating an evaluation distribution based on sorting the co-occurrence distribution with the sorting construct, and determining a set of co-occurrence clusters for the plurality of test types based on the evaluation distribution.

In some embodiments, such as described in the context of the first aspect, the evaluation distribution may be a matrix in which each type of test from the plurality of types of tests corresponds to one row and one column. In some such embodiments, generating the set of co-occurrence clusters based on the evaluation distribution may comprise displaying a representation of the evaluation distribution in which each off-diagonal element of the evaluation distribution is displayed in a cell having a color determined based on relative frequency of co-occurrences for tests of the type corresponding to that off-diagonal element's column with tests of the type corresponding to that off-diagonal element's row. In such embodiments, generating the set of co-occurrence clusters based on the evaluation distribution may also comprise receiving input from a user, the input indicating one or more sections of the evaluation distribution which should be grouped together into co-occurrence clusters.

In some embodiments such as described in the context of the first aspect, generating the set of co-occurrence clusters based on the evaluation distribution may comprise performing a partitioning process on a defined portion of the evaluation distribution. In some embodiments where it is present, such a partitioning process may comprise, for each of a set of one or more types of test taken from the types of tests in the defined portion of the evaluation distribution, determining a connection value associated with partitioning between that type of test and the next type of test from the defined portion of the evaluation distribution. In such embodiments, the partitioning process may further comprise identifying a partition associated with a lowest determined connection value as the partition to apply to the defined portion of the evaluation distribution.

In some embodiments which comprise a partitioning process as described in the preceding paragraph, the partitioning process may comprise, after identifying the partition to apply to the defined portion of the evaluation distribution, determining whether to further partition any sub-portion of the defined portion of the evaluation distribution defined based on the identified partition. In such embodiments, the partitioning process may also comprise, for each sub-portion of the defined portion of the evaluation distribution where a determination is made to further partition that sub-portion, performing the partitioning process with that sub-portion as the defined portion of the evaluation distribution.

In some embodiments which comprise performing the partitioning process with sub-portions of the defined portion of the evaluation distribution such as described in the preceding paragraph, the partitioning process may comprise, for each sub-portion of the defined portion of the evaluation distribution where a determination is made to further partition that sub-portion, before determining connection values associated with partitions in that sub-portion, performing a set of steps for that sub-portion. In some embodiments, such a set of steps may comprise generating a transformation operator based on that sub-portion, generating a sorting construct based on the transformation operator generated based on that sub-portion, and sorting that sub-portion with the sorting construct generated based on the transformation operator generated based on that sub-portion.

In some embodiments of the types described in either of the preceding two paragraphs, determining whether to further partition any sub-portion of the defined portion of the evaluation distribution may comprise comparing a connectedness threshold with a connectedness value between that sub-portion and another sub-portion of the defined portion of the evaluation distribution defined based on the identified partition. In such embodiments, determining whether to further partition any sub-portion of the defined portion of the evaluation distribution may further comprise comparing a size of that sub-portion with a cluster size threshold.

In some embodiments of the type described in the preceding paragraph, a connectedness value may be determined using an equation that combines connectedness metrics for sub-portions of the defined portion of the evaluation distribution defined based on the identified partition.

In some embodiments which comprise a partitioning process comprising performing acts for each of a set of one or more types of tests taken from the types of tests in the defined portion of the evaluation distribution, the set of one or more types of tests taken from the types of tests in the defined portion of the evaluation distribution may comprise each type of test in the defined portion of the evaluation distribution.

In some embodiments such as described in the context of the first aspect, the co-occurrence data may comprise, for each of the plurality of types of tests as a subject test type, for each other type of test from the plurality of types of tests, a number of times tests of the subject test type were included in a single order with tests of that other test type. In some such embodiments, the co-occurrence distribution may be a symmetrical co-occurrence matrix in which each type of test corresponds to one row and one column and each off-diagonal element in the co-occurrence matrix may represent the number of times tests having the test type corresponding to that off-diagonal element's column were included in a single order with tests having the test type corresponding to that off-diagonal element's row. Further in some such embodiments, the transformation operator generated based on the co-occurrence distribution may be a Laplacian matrix, the sorting construct generated based on the transformation operator may be a first nonzero eigenvector of the Laplacian matrix, and the evaluation distribution may be a matrix in which each type of test from the plurality of types of tests corresponds to one row and one column.

Corresponding systems comprising one or more computers configured by computer executable instructions stored on non-transitory computer readable media to perform steps of methods described in any of the preceding embodiments, as well as non-transitory computer readable media storing instructions for performing steps of method described in any of the preceding embodiments, could also be implemented without undue experimentation by those of ordinary skill in the art based on this disclosure. Accordingly, the preceding description of potential embodiments and aspects should be understood as being illustrative only, and should not be treated as limiting.

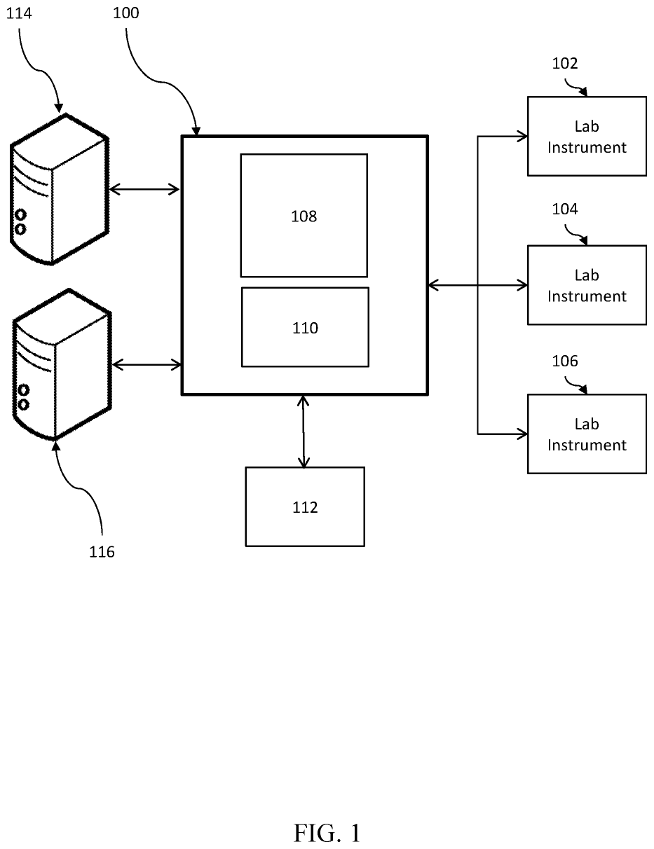

Turning now to the figures, FIG. 1 shows a schematic diagram of an exemplary information system 100 for identifying clusters of tests which have a high frequency of co-occurrence. The exemplary information system 100 may be configured to receive assay data from one or more lab instruments 102 104 106. This assay data may include information such as sample IDs of samples that tests are performed on, as well as the tests that were performed. As shown in FIG. 1, such an information system 100 may include one or more computing servers 108, and one or more memories 110 which may be used, respectively to process and store information received from the one or more lab instruments 102 104 106. Additionally, in some embodiments an information system 100 such as shown in FIG. 1 may also include or be in communication with a display 112 which could be used to provide either intermediate or final results of the information system's processing to a user.

Of course, it should be understood that the description above of an information system 100 obtaining assay data directly from lab instruments 102 104 106 is intended to be illustrative only, and should not be treated as limiting on the scope of protection provided by this document or any other document claiming the benefit of this disclosure. For example, in some embodiments, either in addition to, or as an alternative to, obtaining assay data directly from laboratory instruments, an information system 100 may obtain such data from either laboratory information systems 114 or hospital information systems 116. Similarly, it should be understood that while FIG. 1 illustrates various laboratory instruments 102 104 106 as being connected to the information system 100 via a shared network (e.g., a common LAN), it is possible that, in some embodiments, an information system may collect assay data from laboratory instruments which are not so interconnected (e.g., instruments located at different laboratories that do not share a common network). Similarly, while FIG. 1 illustrates only a single laboratory information system 114 and hospital information system 116, it is possible that some embodiments may collect assay data from multiple laboratory information systems, and/or multiple hospital information systems. Accordingly, while some embodiments may follow the architecture shown in FIG. 1, that architecture should be seen as illustrative only, and should not be treated as limiting.

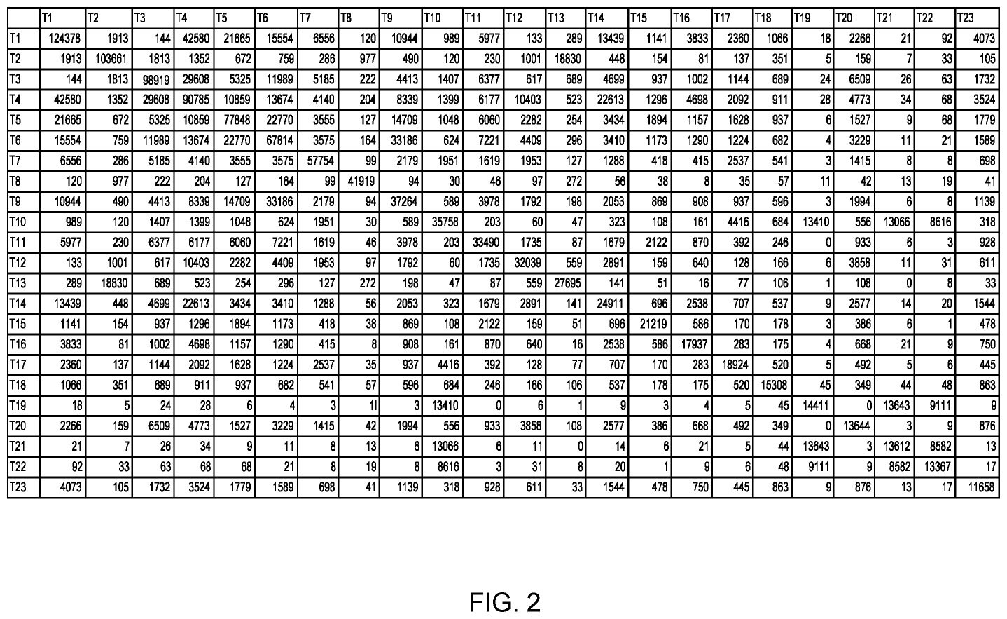

Turning now to FIG. 2, that figure shows a co-occurrence distribution represented as a co-occurrence matrix for generic tests such as could be run on lab instruments 102 104 106 of FIG. 1. In that matrix, the diagonal entries indicate the number of times a particular test was ordered (e.g., test T1 was ordered 123,378 times, test T2 was ordered 103,661 times, etc.). The off-diagonal entries then illustrate the number of times different tests were ordered together. For example, number 1,913 in the second column of the first row and the second row of the first column indicates that tests T1 and T2 were ordered together 1,913 times. As will be apparent, a co-occurrence distribution such as the co-occurrence matrix shown in FIG. 2 may be populated in a variety of manners. For example, in some embodiments an information system 100 such as shown in FIG. 1 may populate a matrix such as shown in FIG. 2 by pulling a subset of the data stored in its memory/memories 110 (e.g., co-occurrence data from the preceding two weeks for the most frequently ordered types of tests). Alternatively, in some embodiments, all assay data from an information system's memory/memories 110 could be used to populate a co-occurrence distribution, thereby providing a more comprehensive view of the data available to the organization maintaining the information system 100. Of course, in some embodiments a user of the information system 100 may be able to specify the data that should be used to populate a co-occurrence distribution, thereby providing flexibility for different population approaches to be used for different purposes (e.g., populating with a recent subset of data for identifying current practices, versus populating with a subset corresponding to a particular past time period to identify differential effects that the policies in place during the historical and more recent time periods may have had). Accordingly, the above described approaches to populating a co-occurrence distribution such as the co-occurrence matrix shown in FIG. 2 should be understood as being illustrative only, and should not be treated as limiting.

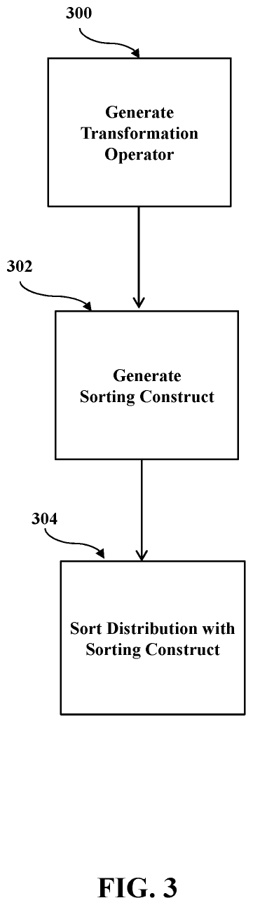

Turning now to FIG. 3, that figure shows a process that, in some embodiments, may be used to derive an evaluation distribution from a co-occurrence distribution such as the co-occurrence matrix shown in FIG. 2. Initially in the process of FIG. 3, a transformation operator (e.g., a derivative operator, such as a Laplacian matrix) will be determined 300 based on the co-occurrence matrix. This may be done by, to use the creation of a Laplacian matrix (also referred to herein as a “Laplacian”) from a co-occurrence matrix as an example, by creating a diagonal matrix D, in which each element D is the sum of the elements from the i^{th} row of the co-occurrence matrix, and then subtracting the co-occurrence matrix to obtain a new matrix L=D−W. After the transformation operator has been determined 300, the process of FIG. 3 continues with generating 302 a sorting construct which, in some embodiments, may be the first non-zero eigenvector (e.g., the eigenvector corresponding to the lowest non-zero eigenvalue) of the transformation operator (e.g., the first non-zero eigenvector of the Laplacian, in embodiments where the operator is a Laplacian matrix).

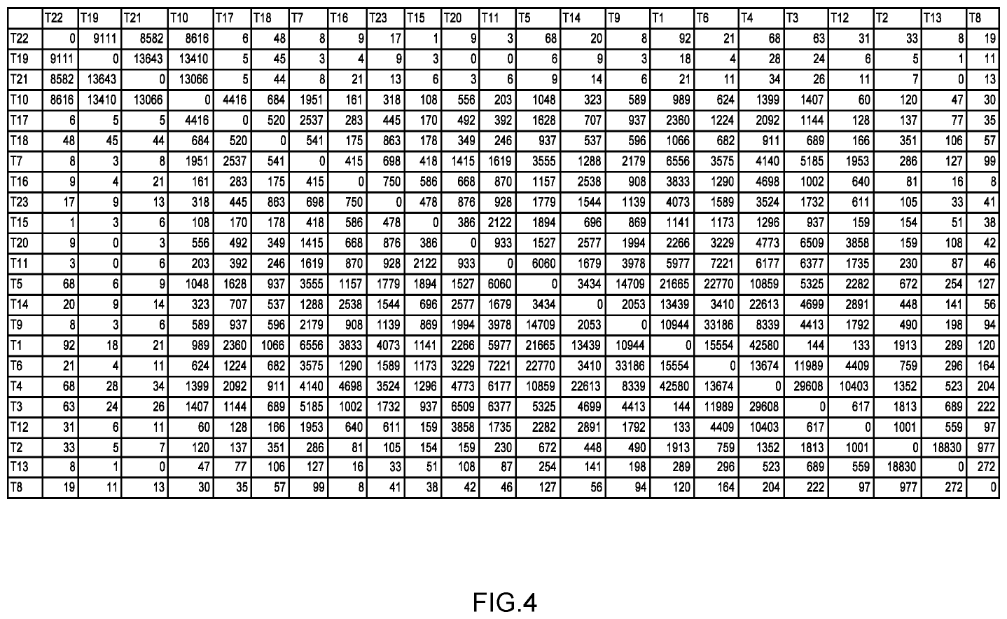

With the sorting construct available, in some embodiments, a process such as shown in FIG. 3 could continue with sorting 304 the co-occurrence distribution using the sorting construct. In some embodiments where the co-occurrence distribution is a co-occurrence matrix and the sorting construct is the first non-zero eigenvalue of the co-occurrence matrix's Laplacian, this sorting 304 could be done by recording the position of each element in the eigenvector, sorting the eigenvector while tracking each element's position, using the relationships between the original and final positions of the eigenvector's elements to create a mapping from the original to the final ordering (e.g., a function ƒ that, for an input integer n representing a position in the sorted eigenvector, returns an integer output showing the position that the n^{th} element in the sorted eigenvector occupied in the unsorted eigenvector), and applying that mapping to the test correlation matrix to obtain a sorted test correlation matrix (e.g., creating a new matrix in which each element Eᵢⱼ is equal to element f(i)f(j) in the unsorted co-occurrence matrix). An example of a sorted matrix such as could be obtained by applying the above steps to the matrix of FIG. 2 is illustrated in FIG. 4. Please note that while the diagonal entries in FIG. 4 are 0 (representing that, in an adjacency graph representation of test co-occurrence, vertices would have edges connecting each other but not themselves), this preferably will not impact the analysis since the co-occurrences are reflected by the off-diagonal elements.

After an evaluation distribution (e.g., a matrix such as shown in FIG. 4) has been derived, in some embodiments the evaluation distribution may be used to identify clusters of tests with high (or relatively high) frequency of co-occurrence. As will be apparent to those of ordinary skill in the art, there are various ways in which this clustering could be performed, and different embodiments may utilize different approaches or combinations of approaches to clustering. For example, in some embodiments, clusters of tests with high co-occurrence may be identified by presenting a matrix representation of the evaluation distribution to a human operator and taking advantage of the fact that the human eye is generally very skilled in identifying visual patterns and groupings. In embodiments which include this type of visual clustering, there may be preparatory steps performed to facilitate identification of groups. For instance, cells in the matrix representation of the evaluation distribution may be colored to show their value (e.g., linearly or logarithmically transitioning from pure green for cells with a value of 0 to pure red to the cells with the highest values) and therefore the co-occurrence of the tests corresponding to the rows and columns of the relevant cells. Other types of preparation may also be performed in some cases. For example, in some embodiments, prior to coloring diagonal elements in a matrix representation of an evaluation distribution may be set equal to the average of their adjacent cells so that they would tend to blend in and enhance clusters rather than bisect and detract from the user's ability to identify them.

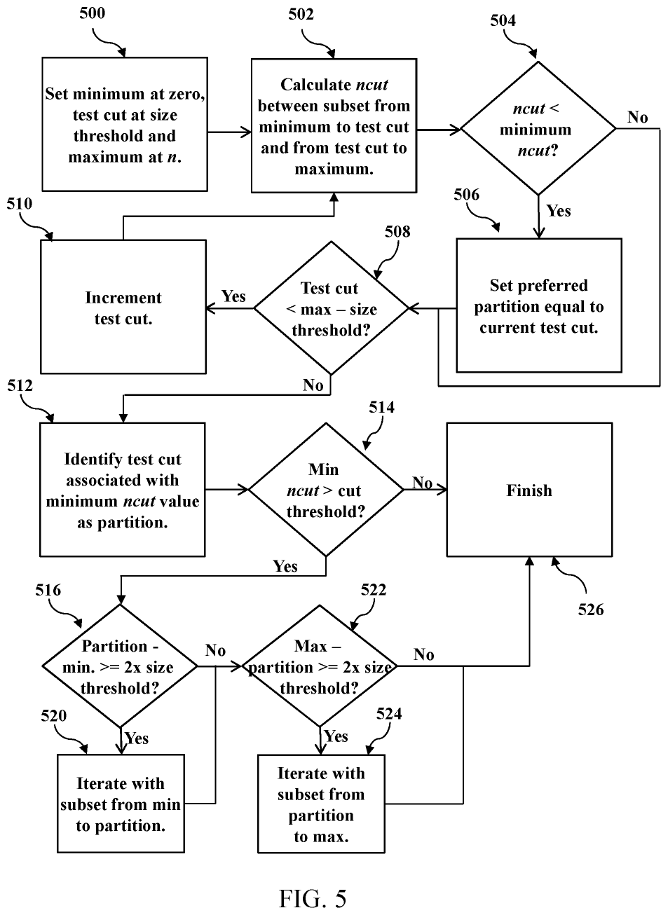

It should be understood that the above description of visually identifying clusters in a matrix representation of an evaluation distribution is intended to be illustrative only, and that other approaches may be used in some embodiments. For example, in some embodiments, a process such as shown in FIG. 5 may be applied to automatically identify clusters in an evaluation distribution such as the sorted analysis matrix shown in FIG. 4. At a high level, the process of FIG. 5 will segment an evaluation distribution into sub-portions (e.g., in the case of a matrix representation of the evaluation distribution, submatrices lying along the original analysis matrix's diagonal) and will then iteratively segment those sub-portions so long as various cluster size and connectivity requirements are met. In more detail, the process of FIG. 5 begins with setting values 500 that will be used in later processing. Specifically, the process of FIG. 5 will set a minimum value (used for defining the upper left corner of the upper left submatrix) at zero, a maximum value (used in embodiments which represent an evaluation distribution in matrix form for defining the lower right corner of the lower right submatrix) at n (e.g., the number of rows/columns in an analysis matrix), and a test cut value (used to determine where to partition the evaluation distribution, such as a location along an analysis matrix's diagonal) equal to a sub-portion size threshold (a parameter defining the minimum size for a cluster, which will preferably be set by a user, but may also be set automatically as a percentage of the total number of elements in the evaluation distribution, the total number of rows/columns in a matrix representation of an evaluation distribution, or as a constant default value).

With the analysis values having been set 500, a process such as shown in FIG. 5 will continue with calculating 502 an ncut value between subsets of elements defined by the test cut value. The ncut value can be seen as a measure of how connected the portions of the evaluation distribution are to each other. In embodiments where the evaluation distribution takes the form of an analysis matrix such as shown in FIG. 4, this can be calculated using equation 1, below:

$$ncut ( A , B ) = \frac{c ( A , B )}{c ( A , A + B )} + \frac{c ( A , B )}{c ( B , A + B )} \\ ncut value calculation \qquad Equation 1$$

In equation 1, A and B represent subsets of row/columns (which can be seen as interchangeable since the analysis matrix is symmetric) that would be divided by the test cut value and c(A, B) is the sum of the connections between subset A and subset B. Thus, with the analysis matrix of FIG. 4, if the test cut value is three, then subset A would be the three rows corresponding to tests T22, T19 and T21, and subset B would be the 20 rows starting with the row representing test T10 and continuing through the row representing test T8). c(A, B) could then be found by summing the relevant elements in those rows using equation 2, below:

$$c ( A , B ) = {\sum }_{i ∈ A , j ∈ B} {w}_{ij} \\ exemplary connectivity measure equation \qquad Equation 2$$

In the process of FIG. 5, after the ncut value has been calculated 502, it is compared 504 with the smallest previously calculated ncut value (or, in some embodiments, if no previous ncut value has been calculated for the current minimum and maximum parameters, this step may be skipped). If the ncut value for the current test cut is less than the smallest previously calculated ncut value, then the current test cut can be identified 506 as the preferred partition, reflecting the fact that it is the best way (so far) identified for separating the elements defined by the maximum and minimum values. After the ncut value has been checked 504 and the preferred partition value updated 506 (if needed), a further check 508 can be made of whether the current test cut value is greater than the previously set maximum size minus the previously defined sub-portion size threshold. In a process such as shown in FIG. 5, this type of test 508 could be used to prevent needlessly checking partitions that would create sub-portions that are smaller than the previously defined threshold size. Then, if the test cut value was greater than the maximum value minus the size threshold, the test cut value could be incremented 510 and the process could return to calculating 502 the ncut value for the new test cut value, and this type of iteration could be repeated until the entire diagonal of the analysis matrix (less the left and right portions which were less than the size threshold) had been traversed.

After all potential partitions between the minimum and maximum values (e.g., after the diagonal of an analysis matrix from the minimum to maximum value (less thresholds) had been traversed) the test cut associated with the minimum ncut value would be identified 512 as the value to use for partitioning the elements between the previously set minimum and maximum values. At this point, in some embodiments a set of one or more checks might be performed to determine if further divisions should be made in either of the sub-portions. For example, in some embodiments, a predefined connectedness threshold may have been set such that a check 514 showing the ncut value did not exceed that threshold would be treated as indicating that no further partitioning between the maximum and minimum values was necessary. Similarly, in some embodiments, a check 516 could be performed to confirm if the subset from the minimum value to the partition was at least twice the minimum cluster size. Then, if it was, the process could iterate 518 by partitioning that subset (e.g., by leaving the minimum unchanged, setting the maximum to the current partition value, and returning to the previously described calculation 502 of ncut). Similarly, in some embodiments a check 522 may be performed if the subset from the partition to the maximum value was at least twice the minimum cluster size. Then, if it was, the process could iterate 524 by partitioning that subset (e.g., by setting the minimum equal to the current partition value, leaving the maximum unchanged, and returning to the previously described calculation 502 of ncut). Finally, in the process of FIG. 5, once the various checks (e.g., the checks 514, 516 and/or 522 shown in FIG. 5) indicated that no further partitioning was needed, the process could finish 526, and the various subsets defined by the identified partitions could be treated as the clusters for the evaluation distribution.

Of course, it should be understood that the above description is intended to be illustrative only, and that numerous other embodiments are possible and could be implemented without undue experimentation based on this disclosure by those of ordinary skill in the art. For example, in some embodiments, rather than treating all subsets identified using partitions as separate clusters of tests, a further check could be performed on each subset testing whether the connectedness of its elements relative to elements in the rest of the evaluation distribution (e.g., using the ncut calculation described above, potentially, but not necessarily, with a different threshold than was used for initially determining whether to continue iteration) was sufficient to justify treating it as a cluster that might be worthy of further study. Similarly, in some embodiments, rather than testing for size thresholds before deciding whether to iterate, or limiting iteration to sections of an evaluation distribution limited by a cluster size threshold, testing for size thresholds may be performed subsequently—such as in determining whether a test cut should be treated as a partition used in further analysis.

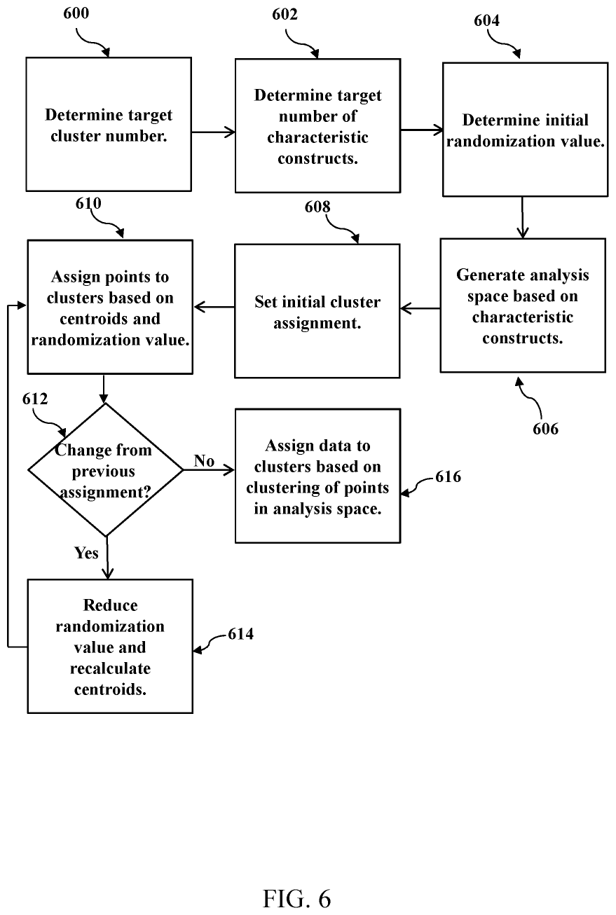

Other types of variations, including variations with diverge from the basic framework depicted in FIG. 3, are also possible. As an example, consider the process illustrated in FIG. 6, which may be used in some embodiments. Initially, in the process of FIG. 6, a determination 600 would be made of a target number of clusters that the data should be organized into (e.g., by asking a user to input a target number of clusters), and a corresponding determination 602 would be made of that number of characteristic constructs (e.g., eigenvectors of an operator determined based on the co-occurrence matrix, such as a co-occurrence matrix's Laplacian). For instance, in some embodiments, if the first determination 600 was that the data should be organized into k clusters, then the second determination 602 would preferably be of the first k eigenvectors for the Laplacian. In some embodiments, a determination 604 may also be made of a randomization value (e.g., by setting the randomization to a default value, such as 1, or to a value proportionate to the maximum elements in the distribution, such as 10% of the absolute value of an analysis matrix's largest element) that could be used to reduce the risk that the clustering algorithm would fall into a sub-optimal local minimum.

After the target number of characteristic constructs had been determined 602 (and, in some embodiments, potentially before determination 604 of an initial randomization value), a process such as shown in FIG. 6 may proceed with generating 606 an analysis space based on those characteristic constructs. In some embodiments where the characteristic constructs are eigenvectors of a co-occurrence matrix's Laplacian, this may be done by assembling the eigenvectors into an analysis matrix in which each eigenvector was a column of the analysis matrix. An initial cluster assignment could then be set 608 for the analysis space. For example, in some embodiments, this could be done by treating each row of an analysis matrix as a point in k-dimensional space (where k is the previously determined target cluster number), and then randomly assigning each of those points to one of k clusters. Alternatively, in some embodiments, initial clusters could be set by randomly choosing k rows of an analysis matrix and treating them as cluster centroids. Other approaches to initially assigning clusters, such requesting a user to make a best guess of how rows in an analysis matrix should be grouped into clusters are also possible, and could be implemented without undue experimentation by those of ordinary skill in the art in light of this disclosure.

In some embodiments which implement a method such as depicted in FIG. 6, after the initial values had been set/determined, points in the analysis space could be assigned 610 to clusters based on the then current centroids and the randomization values. This could be done, for example, by performing a calculation that treats each row in an analysis matrix as a point in k dimensional space, and then measures the distance (e.g., the Euclidian distance, though other distance measures may be used in some embodiments) between that point and the locations of the centroids for the then current clusters. The points (e.g., the rows in the analysis matrix) could then be assigned to the clusters with the closest centroids. Additionally, in some embodiments the distances may be modified using the randomization value. For example, for a point p with distances d₁, d₂, . . . dₖ from the k centroids, each of the distances might be randomly modified based on the randomization value (e.g., multiplied by the product of the randomization value and a random number between −1 and 1) before point p was assigned to a cluster. In this way, some embodiments may reduce the risk that their cluster assignments will become trapped at a local minimum, since the additional randomization might introduce enough noise to break out of a local minimum once it was entered.

After the points in the analysis space had been assigned 610 to clusters, in some embodiments, a method such as depicted in FIG. 6 could check to see if that assignment was different from the preceding assignment. For instance, the initial cluster assignment was set 608 randomly, the check 612 would examine whether any of the points were in different clusters than the ones to which they had initially been randomly assigned. If any assignments had changed, then an update 614 could be performed by reducing the randomization value (e.g., dividing it by 10) and recalculating the cluster centroids using the new cluster assignments. The process could then iterate by repeating the assignment 610 step, checking for changes 612, and continuing until the assignments stabilized and no changes were detected. At this point, the clustering could be deemed to be complete and the underlying data could be assigned 616 to clusters based on the clustering of the points in the analysis space (e.g., if row 1 of an analysis matrix representation of k dimensional analysis space was assigned to cluster 1, then the test type corresponding to row 1 of the underlying co-occurrence matrix could be assigned to cluster 1, if row 2 of the analysis matrix was assigned to cluster 3 then the test type corresponding to row 2 of the underlying co-occurrence matrix could be assigned to cluster 3, etc.).

Further variations on, and features for, the inventors' technology will be immediately apparent to, and could be practiced without undue experimentation by, those of ordinary skill in the art in light of this disclosure. For example, in some embodiments which utilize k-means clustering as described in the context of FIG. 6, the k-means clustering may be applied directly to a co-occurrence matrix (or other representation of a co-occurrence distribution), rather than to a derived construct such as rows in an analysis matrix. Similarly, in some embodiments identification of clusters using k-means clustering may be part of a larger process in which a user would specify a target cluster number, be presented with that number of clusters determined using k-means clustering, and then be able to repeat with a new target cluster number and compare the results to determine final clustering for the test types. Accordingly, instead of limiting the protection accorded by this document, or by any document which is related to this document, to the material explicitly disclosed herein, the protection should be understood to be defined by the claims, if any, set forth herein or in the relevant related document when the terms in those claims which are listed below under the label “Explicit Definitions” are given the explicit definitions set forth therein, and the remaining terms are given their broadest reasonable interpretation as shown by a general purpose dictionary. To the extent that the interpretation which would be given to such claims based on the above disclosure is in any way narrower than the interpretation which would be given based on the “Explicit Definitions” and the broadest reasonable interpretation as provided by a general purpose dictionary, the interpretation provided by the “Explicit Definitions” and broadest reasonable interpretation as provided by a general purpose dictionary shall control, and the inconsistent usage of terms in the specification or priority documents shall have no effect.

### Explicit Definitions

When appearing in the claims, a statement that something is “based on” something else should be understood to mean that something is determined at least in part by the thing that it is indicated as being “based on.” When something is required to be completely determined by a thing, it will be described as being “based exclusively on” the thing.

When used in the claims, “determining” should be understood to refer generating, selecting, defining, calculating or otherwise specifying something. For example, to obtain an output as the result of analysis would be an example of “determining” that output. As a second example, to choose a response from a list of possible responses would be a method of “determining” a response. As a third example, to identify data received from an external source (e.g., a microphone) as being a thing would be an example of “determining” the thing.

When used in the claims a “means for automatically identifying co-occurrence clusters from tests performed on one or more laboratory instruments” should be understood as a means plus function limitation as provided for in 35 U.S.C. § 112(f), in which the function is “automatically identifying co-occurrence clusters from tests performed on one or more laboratory instruments” and the corresponding structure is a computer configured to perform processes as illustrated in FIG. 5 and described in the corresponding text.

1. A method comprising:
a) obtaining a set of co-occurrence data for each of a plurality of types of tests performed on patient samples;
b) defining a co-occurrence distribution based on the set of co-occurrence data;
c) determining a transformation operator based on the co-occurrence distribution;
d) determining a sorting construct based on the transformation operator;
e) generating an evaluation distribution based on sorting the co-occurrence distribution using the sorting construct determined based on the transformation operator; and
f) generating a set of co-occurrence clusters for the plurality of types of tests based on the evaluation distribution.

2. The method of claim 1, wherein:
a) the evaluation distribution is a matrix in which each type of test from the plurality of types of tests corresponds to one row and one column;
b) generating the set of co-occurrence clusters based on the evaluation distribution comprises:
i) displaying a representation of the evaluation distribution in which each off-diagonal element is displayed in a cell having a color determined based on relative frequency of co-occurrences for tests of the type corresponding to that off-diagonal element's column with tests of the type corresponding to that off-diagonal element's row; and
ii) receiving input from a user, the input indicating one or more sections of the evaluation distribution which should be grouped together into co-occurrence clusters.

3. The method of claim 1, wherein generating the set of co-occurrence clusters based on the evaluation distribution comprises performing a partitioning process on a defined portion of the evaluation distribution, wherein the partitioning process comprises:
a) for each of a set of one or more types of tests taken from the types of tests in the defined portion of the evaluation distribution, determining a connection value associated with partitioning between that type of test and the next type of test from the defined portion of the evaluation distribution; and
b) identifying a partition associated with a lowest determined connection value as the partition to apply to the defined portion of the evaluation distribution.

4. The method of claim 3, wherein the partitioning process comprises:
a) after identifying the partition to apply to the defined portion of the evaluation distribution, determining whether to further partition any sub-portion of the defined portion of the evaluation distribution defined based on the identified partition; and
b) for each sub-portion of the defined portion of the evaluation distribution where a determination is made to further partition that sub-portion, performing the partitioning process with that sub-portion as the defined portion of the evaluation distribution.

5. The method of claim 4, wherein the partitioning process comprises, for each sub-portion of the defined portion of the evaluation distribution where a determination is made to further partition that sub-portion, before determining connection values associated with partitions in that sub-portion:
a) determining a transformation operator based on that sub-portion;
b) determining a sorting construct based on the transformation operator determined based on that sub-portion; and
c) sorting that sub-portion with the sorting construct determined based on the transformation operator determined based on that sub-portion.

6. The method of claim 4, wherein determining whether to further partition any sub-portion of the defined portion of the evaluation distribution comprises:
a) comparing a connectedness value between that sub-portion and another sub-portion of the defined portion of the evaluation distribution defined based on the identified partition with a connectedness threshold; and
b) comparing a size of that sub-portion with a cluster size threshold.

7. The method of claim 6 wherein the connectedness value is determined using an equation that combines connectedness metrics for sub-portions of the defined portion of the evaluation distribution defined based on the identified partition.

8. The method of claim 3 wherein the set of one or more types of tests taken from the types of tests in the defined portion of the evaluation distribution comprises each type of test in the defined portion of the evaluation distribution.

9. The method of claim 1, wherein:
a) the co-occurrence data comprises, for each of the plurality of types of tests as a subject test type:
i) for each other type of test from the plurality of types of tests, a number of times tests of the subject test type were included in a single order with tests of that other test type;
b) the co-occurrence distribution is a symmetrical co-occurrence matrix, wherein:
i) each type of test corresponds to one row and one column in the co-occurrence matrix;
ii) each off-diagonal element in the co-occurrence matrix represents the number of times tests having the test type corresponding to that off-diagonal element's column were included in a single order with tests having the test type corresponding to that off-diagonal element's row;
c) the transformation operator generated based on the co-occurrence distribution is a Laplacian matrix and the sorting construct generated based on the transformation operator is a first nonzero eigenvector of the Laplacian matrix; and
e) the evaluation distribution is a matrix in which each type of test from the plurality of types of tests corresponds to one row and one column.

10. A system comprising one or more computers configured by computer executable instructions stored on a non-transitory computer readable medium to perform steps comprising:
a) obtaining a set of co-occurrence data for each of a plurality of types of tests;
b) defining a co-occurrence distribution based on the co-occurrence data;
c) determining a transformation operator based on the co-occurrence distribution;
d) determining a sorting construct based on the transformation operator;
e) generating an evaluation distribution based on sorting the co-occurrence distribution with sorting construct; and
f) generating a set of co-occurrence clusters for the plurality of types of tests based on the evaluation distribution.

11. The system of claim 10, wherein:
a) the evaluation distribution is a matrix in which each type of test from the plurality of types of tests corresponds to one row and one column;
b) generating the set of co-occurrence clusters based on the evaluation distribution comprises:
i) displaying a representation of the evaluation distribution in which each off-diagonal element of the matrix is displayed in a cell having a color determined based on relative frequency of co-occurrences for tests of the type corresponding to that off-diagonal element's column with tests of the type corresponding to that off-diagonal element's row; and
ii) receiving input from a user, the input indicating one or more sections of the evaluation distribution which should be grouped together into co-occurrence clusters.

12. The system of claim 10, wherein generating the set of co-occurrence clusters based on the evaluation distribution comprises performing a partitioning process on a defined portion of the evaluation distribution, wherein the partitioning process comprises:
a) for each of a set of one or more types of tests taken from the types of tests in the defined portion of the evaluation distribution, determining a connection value associated with partitioning between that type of test and the next type of test from the defined portion of the evaluation distribution; and
b) identifying a partition associated with a lowest determined connection value as the partition to apply to the defined portion of the evaluation distribution.

13. The system of claim 12, wherein the partitioning process comprises:
a) after identifying the partition to apply to the defined portion of the evaluation distribution, determining whether to further partition any sub-portion of the defined portion of the evaluation distribution defined based on the identified partition; and
b) for each sub-portion of the defined portion of the evaluation distribution where a determination is made to further partition that sub-portion, performing the partitioning process with that sub-portion as the defined portion of the evaluation distribution.

14. The system of claim 13, wherein the partitioning process comprises, for each sub-portion of the defined portion of the evaluation distribution where a determination is made to further partition that sub-portion, before determining connection values associated with partitions in that sub-portion:
a) determining a transformation operator based on that sub-portion;
b) determining a sorting construct based on the transformation operator determined based on that sub-portion; and
c) sorting that sub-portion with sorting construct determined based on the transformation operator determined based on that sub-portion.

15. The system of claim 13, wherein determining whether to further partition any sub-portion of the defined portion of the evaluation distribution comprises:
a) comparing a connectedness value between that sub-portion and another sub-portion of the defined portion of the evaluation distribution defined based on the identified partition with a connectedness threshold; and
b) comparing a size of that sub-portion with a cluster size threshold.

16. The system of claim 15 wherein the connectedness value is determined using an equation that combines connectedness metrics for sub-portions of the defined portion of the evaluation distribution defined based on the identified partition.

17. The system of claim 12 wherein the set of one or more types of tests taken from the types of tests in the defined portion of the evaluation distribution comprises each type of test in the defined portion of the evaluation distribution.

18. The system of claim 10, wherein:
a) the co-occurrence data comprises, for each of the plurality of types of tests as a subject test type:
i) for each other type of test from the plurality of types of tests, a number of times tests of the subject test type were included in a single order with tests of that other test type;
b) the co-occurrence distribution is a symmetrical co-occurrence matrix, wherein:
i) each type of test corresponds to one row and one column in the co-occurrence matrix;
ii) each off-diagonal element in the co-occurrence matrix represents the number of times tests having the test type corresponding to that off-diagonal element's column were included in a single order with tests having the test type corresponding to that off-diagonal element's row;
c) the transformation operator generated based on the co-occurrence distribution is a Laplacian matrix;
d) the sorting construct generated based on the transformation operator is the first nonzero eigenvector of the Laplacian matrix; and
e) the evaluation distribution is a matrix in which each type of test from the plurality of types of tests corresponds to one row and one column.

19. The system of claim 1, wherein the system comprises one or more laboratory instruments in communication with the one or more computers, wherein the one or more laboratory instruments store data corresponding to the set of co-occurrence data.

20. A machine comprising:
a) a means for automatically identifying co-occurrence clusters from tests performed on one or more laboratory instruments; and
b) the one or more laboratory instruments.
